(19) United States
(12) Patent Application Publication
(10) Pub. No.: US 2024/0286332 A1
(43) Pub. Date: Aug. 29, 2024

(54) Barrier Coated Stopper and Method of Forming Same

(71) Applicant: Becton, Dickinson and Company, Franklin Lakes, NJ (US)

(72) Inventors: Alfred W. Prais, Hewitt, NJ (US); Nestor Rodriguez San Juan, Hamburg, NJ (US)

(21) Appl. No.: 18/037,786

(22) PCT Filed: Nov. 19, 2021
(86) PCT No.: PCT/US21/60005

§ 371 Date: May 19, 2023

Related U.S. Application Data
(60) Provisional application No. 63/116,533, filed on Nov. 20, 2020.

(51) Int. Cl. B29C 45/44; A61M 5/315; B29C 45/00; B29C 45/17; B29C 45/26; B29C 45/27; B29C 45/38; B29L 1/00; B29L 31/56

(52) U.S. Cl. CPC B29C 45/4457; A61M 5/31511; B29C 45/0053; B29C 45/1759; B29C 45/2618; B29C 45/2701; B29C 45/382; B29C 2045/0079; B29L 2001/00; B29L 2031/56

(57) ABSTRACT

A method of forming a molded device, such as a stopper, requiring minimal post-molding processing includes providing a four-plate mold assembly (52) having top plate (54), a sprue/vent plate (56) including a core portion (62), a main plate (58) including a recess (66) defining a mold cavity (68) having a contoured surface (70), and a bottom plate (60), closing the mold assembly (52) such that the core portion (62) enters into the recess (66) in the main plate (58) to define a space (72) corresponding to the shape of the stopper (10, 110) to be molded. A polymeric material is introduced into the mold cavity (68) to form the stopper (10, 110). A barrier layer (14, 114) can be inserted between the main plate (58) and bottom plate and adhered to the molded device (10, 110). The plates are opened and the molded device is removed.

### CROSS-REFERENCE TO RELATED APPLICATION

The present application claims priority to U.S. Provisional Application Ser. No. 63/116,533, entitled “Barrier Coated Stopper and Method of Forming Same”, filed Nov. 20, 2020, the entire disclosure of which is hereby incorporated by reference in its entirety.

### BACKGROUND OF THE INVENTION

### Field of the Invention

The present invention is directed to a method of molding a device, such as a stopper for use with a plunger rod within a syringe or a stopper for closing a vial, and more particularly, the invention is directed to a method of molding a stopper using a four-plate, one-shot injection molding device requiring minimal post-molding processing.

### Description of Related Art

Syringe assemblies, in particular hypodermic syringes, are well known in the medical field for dispensing fluids, such as medications. A conventional syringe typically includes an elongate barrel having opposed proximal and distal ends and a chamber there between for receiving the fluid. A passageway extends through the distal end of the syringe barrel and communicates with the chamber. The distal end of the syringe barrel is connected to a needle cannula for delivering fluid from the chamber and passageway. The proximal end of the syringe barrel slidably receives a plunger rod and stopper assembly such that force applied to the plunger rod urges the stopper along the barrel to drive liquid from the chamber through the needle cannula.

A problem with some hypodermic syringes currently on the market involves the amount of liquid which can remain in the barrel after the stopper is advanced the full length of the barrel during the injection process (i.e., the “dead volume”). Thus, there is a need to produce a stopper that is capable of reducing the amount of dead space within a syringe barrel. Various stoppers have been designed for reducing or minimizing dead space within the syringe barrel and that also adequately seal against the internal wall of the syringe barrel. One example of a stopper is disclosed in U.S. Pat. No. 5,795,337, which is incorporated by reference in its entirety. This design comprises a piston-like stopper body for slidable fluid-tight engagement inside a syringe barrel. The body includes a distal end, a proximal end, and a longitudinal axis therethrough. A distally directed, conically-shaped projection is positioned on the distal end of the stopper body. At least one elongate discontinuity, running along the conically-shaped projection is provided. The discontinuities act to prevent unwanted immediate sealing of the projection of the stopper within a passageway extending from the syringe barrel to the needle cannula allowing any trapped fluid within the barrel to run along these discontinuities and into the passageway. Because the stopper is made of resilient material, further pressure on the plunger rod in a distal direction will distort the stopper, squeezing liquid through the passageway, which is being temporarily held open by the discontinuities.

Complex shaped stoppers having a barrier film on the distal face of the stopper are typically molded by a two-shot molding/trimming process. These types of stoppers are currently only used for low volume products where cost is not as critical. A two shot process is roughly two times as expensive as a conventional single shot process. Some commercially available barrier coated stoppers utilize a single shot molding process, however, these stoppers often have undesirable features. One of the undesirable features is that the first rib has to be a trim edge rather than a molded feature and, as such, it cannot be guaranteed to seal the fluid, nor does it ensure an optimal gliding force. Although the stopper includes two additional ribs and solves the container closure integrity (i.e., CCI) risks, such a design is not desirable for certain devices, such as precision injectors, where variable leakage past the first stopper rib affects drug dose accuracy and system hysteresis. Certain stoppers have been designed that surmount these limitations, but have so far only been produced in a manufacturing process that requires two complete molding and trimming steps. Not only is the expense of this production significant, the more steps in the process, the more chance for waste and scrap that exists. Scaling up production of these types of high-precision stoppers using a single shot molding process can result in significant economic advantages.

### SUMMARY OF THE INVENTION

In accordance with one aspect, the present disclosure is directed to a method of molding a device, such as a stopper, using a one-shot injection molding device requiring minimal post-molding processing. The method comprises the steps of providing a four-plate mold assembly having top plate, a sprue/vent plate including a core portion, a main plate including a recess defining a mold cavity having a contoured surface, and a bottom plate, closing the mold assembly such that the core portion enters into the recess in the main plate to define a space corresponding to the shape of the device to be molded, and introducing a polymeric material into said mold cavity to fill said mold cavity and said space to form the molded device. The molded device will have a body and molded surface corresponding to the contoured surface of the mold cavity. The method further comprises cooling the polymeric material, separating the sprue/vent plate from the main plate to withdraw the core portion from within the molded device, followed by separating the sprue/vent plate from the top plate, which is then followed by separating the bottom plate from the main plate, and finally removing molded device or stopper from the main plate.

The step of separating the sprue/vent plate from the main plate causes the vents to break and the core portion to be withdrawn from within the molded device and the main plate. It can be appreciated that the core portion can be designed to have several shapes depending upon the desired internal shape of the device or stopper. For example, the core portion can have a threaded design, a cone-shaped portion with an enlarged portion toward the intersection of the core portion with the sprue/vent plate, a straight sidewall, and the like. Upon opening of the plates of the mold, the molded device can be removed from the main plate by pulling the molded device through the bottom of the main plate.

The step of introducing the polymeric material into the mold cavity comprises injecting a hot polymeric material into the cavity through an injection nozzle and runner associated with the top plate and the step of cooling the mold assembly includes supplying a cooling material to the main plate.

The contoured surface of the main cavity is in the shape of a stopper having a main body defining an open rearward end, a closed front end, and a cylindrical sidewall extending between the open rearward and the closed front end. According to one embodiment, the shape of the contoured surface is configured to create a plurality of ribs extending radially outward around a perimeter of the main body and axially spaced apart along this main body. It can be appreciated that the main cavity can have other contoured surfaces to make other products and/or to create stoppers having different shapes.

According to one embodiment, the bottom plate can have a flat face configured to create a stopper having a flat face at its distal end. According to another embodiment, the bottom plate can include a contoured surface configured to create a stopper having distal end in the shape of a flat portion on an outer edge of the closed front end or distal end of the stopper and extending inward from the outer edge of the stopper and wherein the contoured surface is shaped to create a projection extending from the closed front end, wherein the projection defines a base as it intersects with the distal end of the stopper and extends from the flat portion of the top of the closed front end, said projection being configured to cooperate within a syringe barrel to reduce dead space during injection. It can be appreciated that the bottom plate can have other designs to create stoppers having a variety of shapes at their distal end.

The method can further include the step of providing a film, such as a barrier film, between the bottom plate and the main plate and wherein upon closing of the mold, the film is sandwiched between the bottom plate and the main plate and upon removal of the device from the mold, the film is secured to the molded device. It can be appreciated that the barrier film can be a known film that provides a low friction barrier between, for example, the stopper and a pharmaceutical composition (e.g., a drug, medicine, or other therapeutic material) in the syringe barrel and may inhibit materials from leaching from the stopper or from extraction of compounds from the pharmaceutical composition by the stopper. The method further includes the step of trimming and removing excess film from the molded device.

In accordance with another aspect, the present disclosure is directed to a system for molding a device, such as a stopper, comprising a mold assembly having top plate, a sprue/vent plate, a main plate, and a bottom plate. The sprue/vent plate includes a core portion extending from a surface thereof. It can be appreciated that the core portion can have any shape, depending upon the desired internal shape of the device or stopper being molded. For example, the core portion can have a threaded design, a cone-shaped portion with an enlarged portion toward the intersection of the core portion with the sprue/vent plate, a straight sidewall, and the like. The main plate of the mold includes a recess defining a mold cavity having a contoured surface. This mold cavity is configured for receiving the core portion from the sprue/vent plate to define a space corresponding to the shape of the device to be molded. An injection nozzle and runner are mated to the top plate. The injection nozzle and runner are configured to supply a polymeric material into the mold cavity to fill the space to form the molded device having a body and molded surface corresponding to this contoured surface. At least one cooling member is provided for cooling the polymeric material within the mold cavity.

The system further includes a first mechanism for separating the sprue/vent plate from the main plate to withdraw the core portion from within the molded device, a second mechanism for separating the sprue/vent plate from the top plate, and a third mechanism for separating the bottom plate from the main plate to enable removal of the molded device from the main plate. According to one embodiment, the first, second, and third mechanisms for separating the top plate, sprue/vent plate, main plate, and bottom plate of the mold assembly comprises a series of spring mechanisms of varying force or a series of core lifters to sequentially separating the sprue/vent plate from the main plate; the sprue/vent plate from the top plate; and the bottom plate from the main plate and wherein the spring mechanisms include leader pins to limit the opening of each of the plates.

According to one embodiment, a film can be inserted between the bottom plate and the main plate such that closing of the mold causes the film to be sandwiched between the bottom plate and the main plate to secure the film to the molded device. The system can further include a trim die for removing excess film from the molded device. According to one design, the trim die can comprise a flat faced punch for removing the excess film, however, it can be appreciated that other types of dies having other types of cutting surfaces, such as a razor edge, and/or other types of trimming devices, with or without an air blast, can be used for removing the excess film.

In accordance with yet another aspect, the present disclosure is directed to a stopper adapted for attachment with a plunger rod for use within a syringe barrel. The stopper comprises a main body defining an open rearward end, a closed front end, and a cylindrical sidewall extending between the open rearward end and the closed front end. The open rearward end is adapted to receive a front forward end attachment portion of the plunger rod. The stopper further includes at least one rib extending radially outward around a perimeter of the main body. The at least one rib is configured for forming an active seal with the syringe barrel. A flat portion is provided on a top outer edge surface of the closed front end of the stopper adjacent the at least one rib. The flat portion extends inward from the outer edge of the stopper. The stopper further includes a projection extending from the closed front end, wherein the projection defines a base as it intersects with the distal end of the stopper and extends from the flat portion to the top of the closed front end. The base has a base diameter less than an outside diameter of the distal end of the stopper and the projection has a profile configured to cooperate with an internal surface of the syringe barrel to reduce dead space during injection.

Further examples of the present disclosure will now be described in the following numbered clauses.

Clause 1: A method of molding a device comprising the steps of: providing a mold assembly having top plate, a sprue/vent plate including a core portion, a main plate including a recess defining a mold cavity having a contoured surface, and a bottom plate; closing the mold assembly such that the core portion enters into the recess in the main plate to define a space corresponding to the shape of the device; introducing a polymeric material into said mold cavity to fill said mold cavity and said space to form said molded device having a body and molded surface corresponding to said contoured surface; separating the sprue/vent plate from the main plate to withdraw the core portion from within the molded device; followed by separating the sprue/vent plate from the top plate; followed by separating the bottom plate from the main plate; and removing said molded device from the main plate.

Clause 2: The method according to clause 1, wherein the step of separating the sprue/vent plate from the main plate causes the vents to break and the core portion to be withdrawn from within the molded device and the main plate.

Clause 3: The method according to clauses 1 or 2, wherein the step of removing the molded device from the main plate comprises removing the molded device through the bottom of the main plate.

Clause 4: The method according to any one of clauses 1-3, wherein the step of introducing the polymeric material into the mold cavity comprises injecting a polymeric material into the mold cavity through an injection nozzle and runner associated with the top plate.

Clause 5: The method according to any one of claims 1-4, wherein after the step of introducing polymeric material into the mold cavity, the mold assembly is heated to cure the polymeric material or the mold assembly is cooled by supplying a cooling material to the main plate

Clause 6: The method according to any one of clauses 1-5, wherein the contoured surface of the main cavity is in the shape of a stopper having a main body defining an open rearward end, a closed front end, and a cylindrical sidewall extending between the open rearward and the closed front end.

Clause 7: The method according to clause 6, wherein the shape of the contoured surface is configured to create a plurality of ribs extending radially outward around a perimeter of the main body and axially spaced apart along this main body.

Clause 8: The method according to clauses 6 or 7, wherein the bottom plate has a flat face configured to create a stopper having a flat face or the bottom plate includes a contoured surface configured to create a stopper having a flat portion on an outer edge of a closed front end of the stopper and extending inward from the outer edge of the stopper and wherein the contoured surface is shaped to create a projection extending from the closed front end, wherein the projection defines a base as it intersects with the distal end of the stopper and extends from the flat portion of the top of the closed front end, said projection being configured to cooperate within a syringe barrel to reduce dead space during injection.

Clause 9: The method according to any one of clauses 1-8, including the step of providing a film between the bottom plate and the main plate and wherein upon closing of the mold assembly, the film is sandwiched between the bottom plate and the main plate and upon removal of the device from the mold assembly, the film is secured to the molded device.

Clause 10: The method according to any one of clauses 1-9, including the step of trimming excess film from the molded device.

Clause 11: A system for molding a device comprising: a mold assembly having top plate, a sprue/vent plate, a main plate, and a bottom plate, the sprue/vent plate including a core portion extending from a surface thereof, the main plate including a recess defining a mold cavity having a contoured surface, said mold cavity configured for receiving the core portion from the sprue/vent plate to define a space corresponding to the shape of the device to be molded; an injection nozzle and runner mated to the top plate, said injection nozzle and runner configured for supplying a polymeric material into the mold cavity to fill said space to form said molded device having a body and molded surface corresponding to said contoured surface; at least one cooling member for cooling the polymeric material within the mold cavity; a first mechanism for separating the sprue/vent plate from the main plate to withdraw the core portion from within the molded device; a second mechanism for separating the sprue/vent plate from the top plate; and a third mechanism for separating the bottom plate from the main plate to enable removal of the molded device from the main plate.

Clause 12: The system according to clause 11, wherein the first, second, and third mechanisms for separating the top plate, sprue/vent plate, main plate, and bottom plate of the mold assembly comprises a series of spring mechanisms of varying force or a series of core lifters to sequentially separate the sprue/vent plate from the main plate; the sprue/vent plate from the top plate; and the bottom plate from the main plate and wherein the spring mechanisms include leader pins to limit the opening of each of the plates.

Clause 13: The system according to clauses 11 or 12, wherein a film can be inserted between the bottom plate and the main plate and wherein closing of the mold causes the film to be sandwiched between the bottom plate and the main plate such that the film is secured to the molded device.

Clause 14: The system according to any one of clauses 11-13, including a trim die with a flat faced punch for removing excess film from the molded device and wherein the trim die optionally includes a center having an opening through which a clean air blast can move therethrough to clear trimmed parts from the trim die or a die block.

Clause 15: A stopper adapted for attachment with a plunger rod for use within a syringe barrel, said stopper comprising: a main body defining an open rearward end, a closed front end, and a cylindrical sidewall extending between the open rearward end and the closed front end, a barrier film covering at least the closed front end of the main body; said open rearward end adapted to receive a front forward end attachment portion of the plunger rod; at least one rib extending radially outward around a perimeter of said main body, said at least one rib configured for forming an active seal with the syringe barrel; a flat portion on a top outer edge surface of the closed front end and adjacent the at least one rib, the flat portion extending inward from the outer edge of the stopper; and a projection extending from the closed front end, wherein the projection defines a base as it intersects with the closed front end of the stopper and extends from the flat portion to the top of the closed front end, said base having a base diameter less than an outside diameter of the distal end of the stopper and said projection having a profile configured to cooperate with an internal surface of the syringe barrel to reduce dead space during injection.

Clause 16: The stopper according to clause 15, wherein the flat portion is approximately 0.75-1 mm wide until a start of an upward slope forming the projection.

Clause 17: The stopper according to clauses 15 or 16, wherein the at least one rib comprises a first rib, a second rib, and a third rib, and wherein the second rib has a smaller diameter than the first and third rib.

Clause 18: The stopper according to any one of clauses 15-17, wherein the open rearward end of the stopper includes a cavity configured for receiving a front attachment end of the plunger rod and wherein an inner surface of the cavity includes a plunger rod cavity protrusion extending from a bottom surface of the closed front end of the stopper.

Clause 19: The stopper according to clause 18, wherein the plunger rod cavity protrusion is configured to lightly engage the front attachment end of the plunger rod to increase the force transmitted to the front of the stopper and wherein the plunger rod cavity protrusion has an angled sidewall.

Clause 20: The stopper according to any one of clauses 15-19, wherein the open rearward end of the stopper includes a cavity having a series of threads for threaded attachment with an attachment end of the plunger rod and wherein the threads have a pitch that is slightly decreasing toward a bottom of the plunger rod cavity.

Clause 21: The stopper according to any one of clauses 15-20, wherein a thickness between a roof profile spline AA-BB and an internal roof projection LL is monotonically decreasing and projects upward to point XX.

Clause 22: The stopper according to clause 21, wherein point XX is within the range of 0.25-0.75 mm within a thickness of a stopper wall defined by YY.

### BRIEF DESCRIPTION OF THE DRAWINGS

The above-mentioned and other features and advantages of this disclosure, and the manner of attaining them, will become more apparent and the disclosure itself will be better understood by reference to the following descriptions of embodiments of the disclosure taken in conjunction with the accompanying drawings, wherein:

FIG. 1A is a top perspective view of a stopper for use within a syringe in accordance with an embodiment of the present invention.

FIG. 1B is a side perspective view of the stopper of FIG. 1A in accordance with an embodiment of the present invention.

FIG. 2A is a top perspective view of a stopper for use within a syringe in accordance with an embodiment of the present invention.

FIG. 2B is a cross-sectional side view of the stopper of FIG. 2A in accordance with an embodiment of the present invention.

FIG. 3 is a cross-sectional side view of a four-plate, one-shot injection molding device that can be used to form molded devices, such as the stoppers of FIGS. 1A-1B and 2A-2B in accordance with an embodiment of the present invention.

FIG. 4 is a perspective view of the mold of FIG. 3 in an opened state including the removal of a molded stopper in accordance with an embodiment of the present invention.

FIGS. 5A-5E show the sequential steps of molding a device using the mold of FIG. 3 in accordance with an embodiment of the present invention.

FIG. 6A is a side perspective view of a die punch for cutting excess barrier film after molding of the stopper of FIGS. 1A-1B in accordance with an embodiment of the present invention.

FIG. 6B is a side perspective view of the die punch of FIG. 6A including an air duct for removing the excess film in accordance with an embodiment of the present invention.

FIG. 7A is a side view of the stopper of FIGS. 1A-1B in accordance with an embodiment of the present invention.

FIG. 7B is a cross-sectional view taken along line B-B of the stopper of FIG. 7A in accordance with an embodiment of the present invention.

FIG. 7C is a top view of the stopper of FIG. 7A in accordance with an embodiment of the present invention.

FIG. 7D is a bottom view of the stopper of FIG. 7A in accordance with an embodiment of the present invention.

FIG. 7E is a side view of the stopper of FIG. 7A with a portion of the stopper cut-away to show the interaction of the plunger rod core with the inner portion of the distal end of the stopper in accordance with an embodiment of the present invention.

FIG. 7F is a partial cross-sectional perspective view of the stopper of FIG. 7A showing the inner threads and inner portion of the distal end of the stopper in accordance with an embodiment of the present invention.

FIG. 7G is a cross-sectional view of the stopper of FIG. 7A showing the increasing pitch of the inner threads of the stopper toward the bottom of the plunger rod cavity in accordance with an embodiment of the present invention.

FIG. 8 is a cross-sectional view of a stopper secured to an attachment end of a plunger rod that is formed with the molding device of FIG. 3 in accordance with an embodiment of the present invention.

FIGS. 9A and 9B show the stopper design of FIG. 8 assembled within a syringe barrel before and after application of a distally directed force to the plunger rod and stopper in accordance with an embodiment of the present invention.

FIG. 10A is a representative view illustrating an amount of dead space created within a syringe barrel after application of a distally directed force to the plunger rod using the stopper design of FIGS. 1A-1B in accordance with an embodiment of the present invention.

FIG. 10B is a representative view illustrating an amount of dead space created within a syringe barrel after application of a distally directed force to the plunger rod using the stopper design of FIGS. 2A-2B in accordance with an embodiment of the present invention.

FIG. 11 is a perspective cross-sectional view of a stopper in accordance with an embodiment of the present invention.

Corresponding reference characters indicate corresponding parts throughout the several views. The exemplifications set out herein illustrate exemplary embodiments of the disclosure, and such exemplifications are not to be construed as limiting the scope of the disclosure in any manner.

### DESCRIPTION OF THE INVENTION

The following description is provided to enable those skilled in the art to make and use the described embodiments contemplated for carrying out the invention. Various modifications, equivalents, variations, and alternatives, however, will remain readily apparent to those skilled in the art. Any and all such modifications, variations, equivalents, and alternatives are intended to fall within the spirit and scope of the present invention.

For purposes of the description hereinafter, the terms “upper”, “lower”, “right”, “left”, “vertical”, “horizontal”, “top”, “bottom”, “lateral”, “longitudinal”, and derivatives thereof shall relate to the concept as it is oriented in the drawing figures. However, it is to be understood that the concept may assume various alternative variations, except where expressly specified to the contrary. It is also to be understood that the specific devices illustrated in the attached drawings, and described in the following specification, are simply exemplary embodiments of the concept. Hence, specific dimensions and other physical characteristics related to the embodiments disclosed herein are not to be considered as limiting.

Also, for purposes of the description of the present invention, the term “distal end” is intended to refer to the end of the syringe from which the needle projects and the end of the stopper which is closer to the syringe needle, whereas the term “proximal end” is intended to refer to the end of the syringe closer to the holder of the syringe and furthest from the needle tip and the end of the stopper furthest from the needle tip.

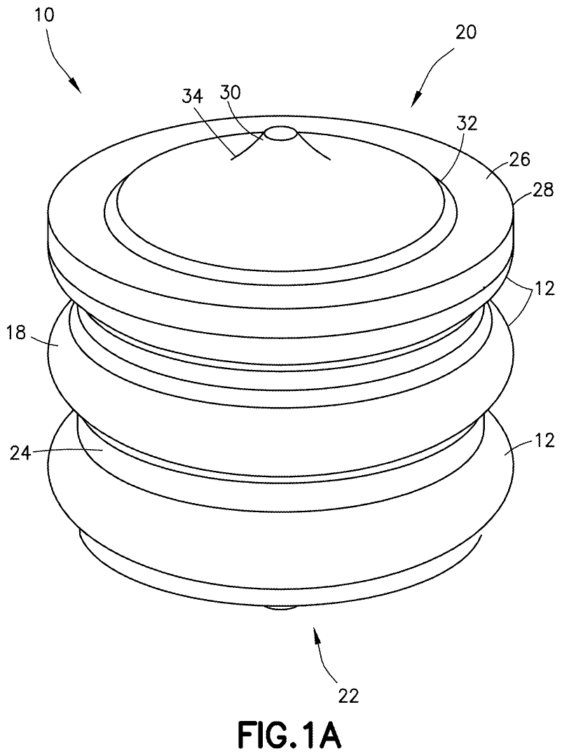

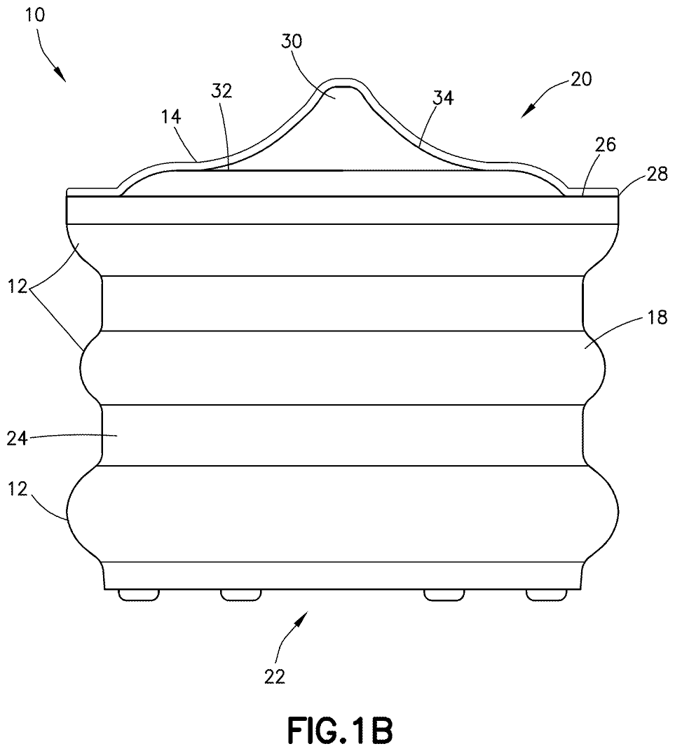

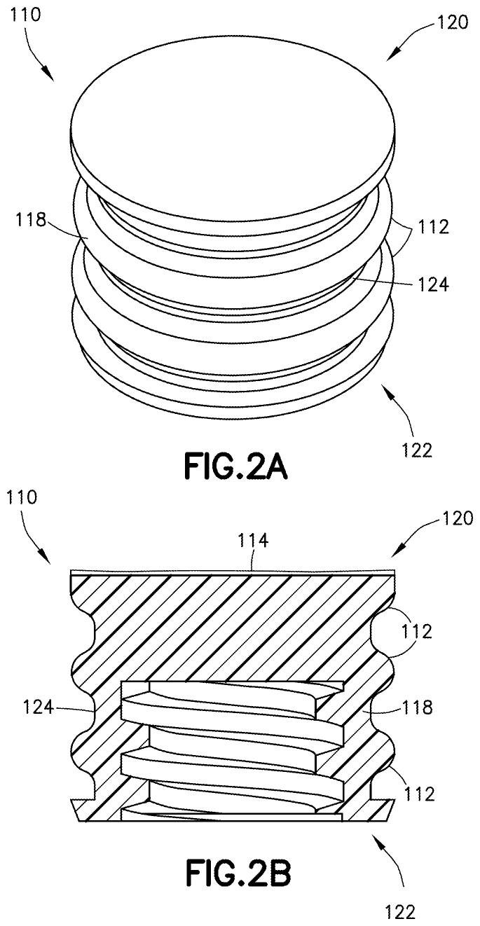

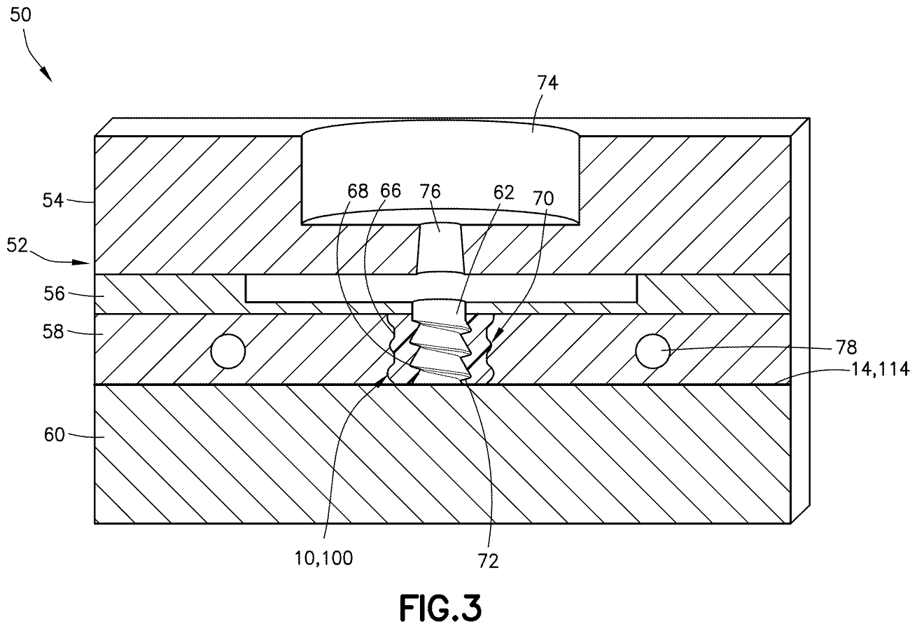

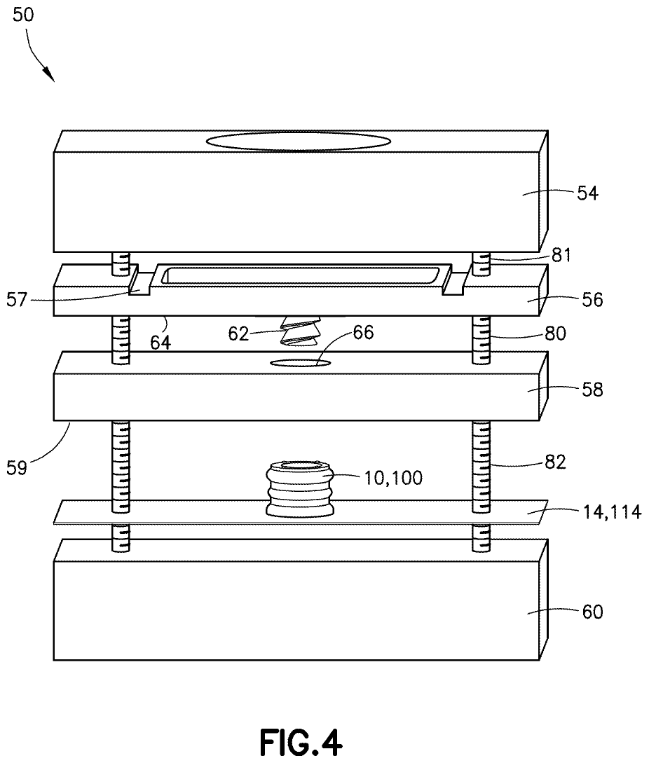

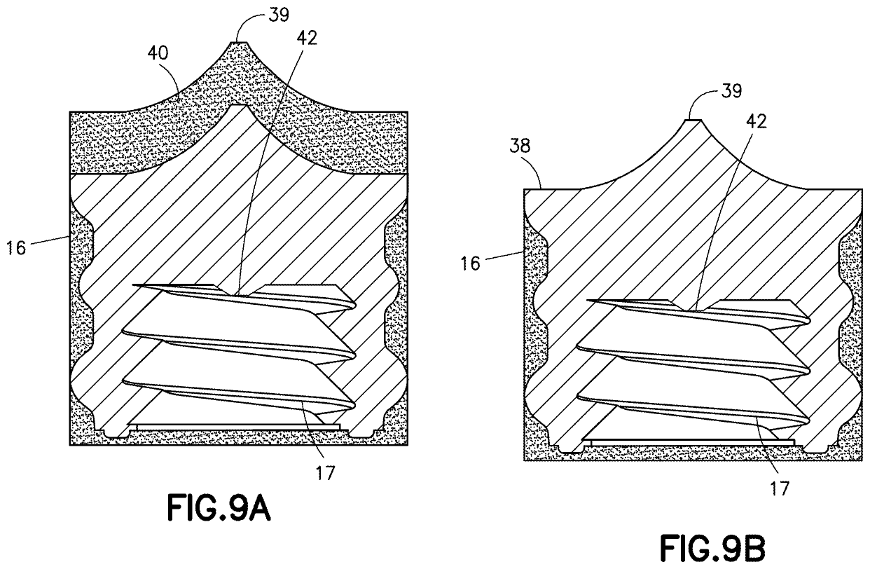

Reference is now made to FIGS. 1A-1B and 2A-2B which show two different types of stoppers 10, 110 which can be produced with the four-plate, one-shot injection molding system, generally indicated as 50, in FIGS. 3 and 4 of the present disclosure. The stopper can include a plurality of annular ribs 12, 112 configured for sealing with fluid-tight engagement with an inside surface of a syringe barrel 16, shown in FIGS. 9A1-9E2.

A syringe barrel typically includes an open proximal end, distal end 38, as shown in FIGS. 9A and 9B, and a cylindrical body portion there between defining a chamber for retaining liquid, such as liquid medication. The syringe barrel can be made of glass, plastic, or a combination thereof, and may include a flange at its proximal end. Examples of plastic materials that can be used to form the syringe barrel include, but are not limited to, substantially transparent thermoplastic such materials as polycarbonate, polypropylene, polyethyleneterephalate (PET) and the like.

The stopper 10, 110 can be positioned in fluid-tight engagement inside the barrel through the action of annular ribs 12, 112 with the distal end 20, 120 of the stopper 10, 110 facing the distal end of the syringe barrel. The distal end 38 of the syringe barrel includes a passageway 39 which is in fluid communication with the chamber. An elongated needle cannula having a lumen extending therethrough, not shown, can be provided for connection to the distal end 38 of the barrel so that the lumen is in fluid communication with the chamber through passageway. It can be appreciated that the needle can be permanently attached to the syringe barrel, such as through the use of adhesives, or it can be removably attached to the syringe barrel, such as through a needle hub, which is permanently attached to the needle cannula and frictionally attached to the tip at the distal end of the syringe barrel. The stopper 10, 110 will be able to slide in the chamber for initial positioning adjacent a drug and for subsequently urging a drug through the lumen of the needle cannula or through the passageway of the distal end of the syringe barrel 38. Alternatively, the stopper 10, 110 can slide within the syringe barrel for reconstitution purposes.

A barrier film 14, 114 can be provided on a distal end 20, 120 of the stopper to reduce and/or eliminate direct contact of the material of the stopper 10, 110 and the syringe contents and to reduce friction between the syringe contents and the stopper 10, 110.

With continuing reference to FIGS. 3 and 4, there is shown the four-plate injection molding system 50. Although the molding system is directed to molding a stopper for use with a plunger rod for a syringe, it can be appreciated that the molding system can be used for molding other products, such as closures for medical vials or test tubes, or other known molded devices. The molding system 50 comprises a mold assembly 52 having top plate 54, a sprue/vent plate 56, a main plate 58, and a bottom plate 60. The sprue/vent plate 56 includes a core portion 62 extending from a bottom surface 64 thereof. It can be appreciated that the core portion 62 can have any shape, depending upon the desired internal shape of the device or stopper 10, 110 being molded. For example, the core portion 62 can have a threaded design, a cone-shaped portion with an enlarged portion toward the intersection of the core portion with the sprue/vent plate, a straight sidewall, and the like. The main plate 58 of the mold includes a recess 66 defining a mold cavity 68 having a contoured surface 70. This mold cavity 68 is configured for receiving the core portion 62 from the sprue/vent plate 56 to define a space 72 corresponding to the shape of the device to be molded. An injection nozzle 74 and runner 76 are mated to the top plate 54. The injection nozzle 74 and runner 76 are configured the supply a polymeric material into the mold cavity 68 to fill the space 72 to form the molded device having a body and molded surface corresponding to the contoured surface 70. At least one cooling member 78 is provided for cooling the polymeric material within the mold cavity 58. The cooling member 78 can be a series of tubes including a cooling medium flowing therethrough or any other known cooling member.

The system 50 further includes a first mechanism 80 for separating the sprue/vent plate 56 from the main plate 58 to withdraw the core portion 62 from within the molded device 10, 110, a second mechanism 81 for separating the sprue/vent plate 56 from the top plate 54 and a third mechanism 82 for separating the bottom plate 60 from the main plate 58 to enable removal of the molded device 10, 110 from the main plate 58. According to one embodiment, the first, second, and third mechanisms 80, 81, 82 for separating the top plate, sprue/vent plate, main plate, and bottom plate of the mold assembly comprises a series of spring mechanisms of varying force to sequentially separate the sprue/vent plate 56 from the main plate 58; the sprue/vent plate 56 from the top plate 54; and the bottom plate 60 from the main plate 58. The spring mechanisms can include leader pins to limit the opening of each of the plates. It can also be appreciated that the first, second and third mechanisms 80, 81, 82 for separating the top plate, sprue/vent plate, main plate, and bottom plate of the mold assembly can comprise a series of core lifters.

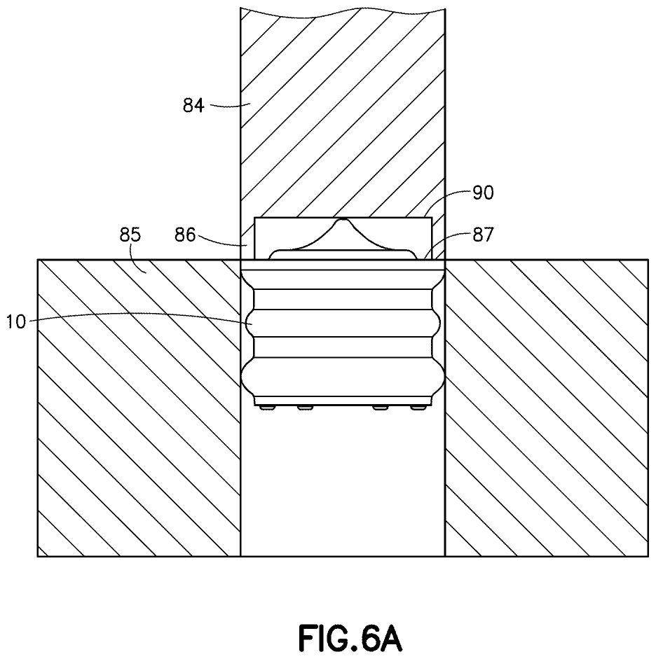

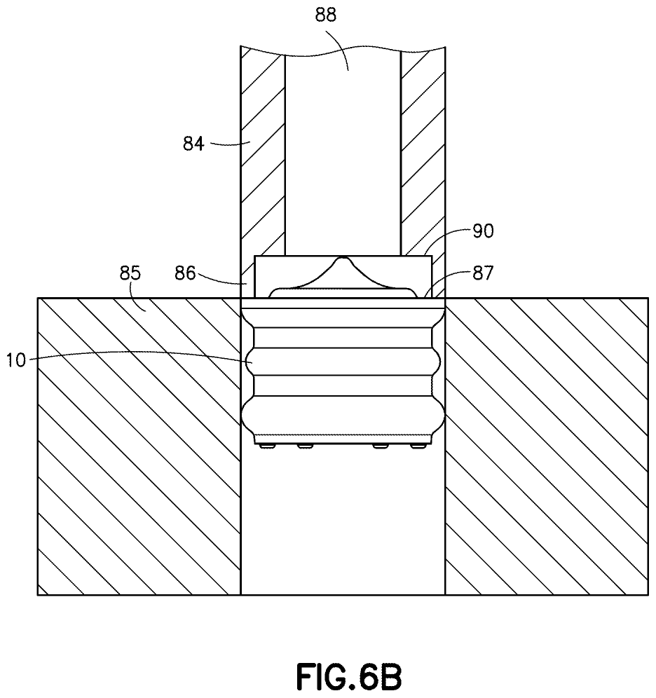

According to one embodiment, a barrier film 14, 114 can be inserted between the bottom plate 60 and the main plate 58 such that closing of the mold assembly 52 causes the film 14, 114 to be sandwiched between the bottom plate 60 and the main plate 58 to secure the film 14, 114 to the molded 10, 110. The use of this barrier film 14, 114 on a stopper improves the sliding action of the stopper within the syringe barrel. The barrier film 14, 114 should not exhibit detrimental effects to the drug and should be free of silicone-based materials, such as silicone oil. One example of barrier films 14, 114 that can be used are fluoropolymer laminates, which are known to have good biocompatibility, good mechanical integrity, are inert, and are processable. The superior strength of the expanded fluoropolymer structure allows these materials to form thin barriers, which remain intact during the forming process and installation of the stopper into the syringe barrel. For example, the barrier film may be selected from the group consisting of polytetrafluoroethylene resin (PTFE), an ethylenetetrafluoroethylene resin (ETFE), expanded polytetrafluoroethylene (ePTFE) and the like. A trim die 84, discussed in further detail below, and shown in FIGS. 6A and 6B, can be provided to remove the excess barrier film 14, 114 from the molded device.

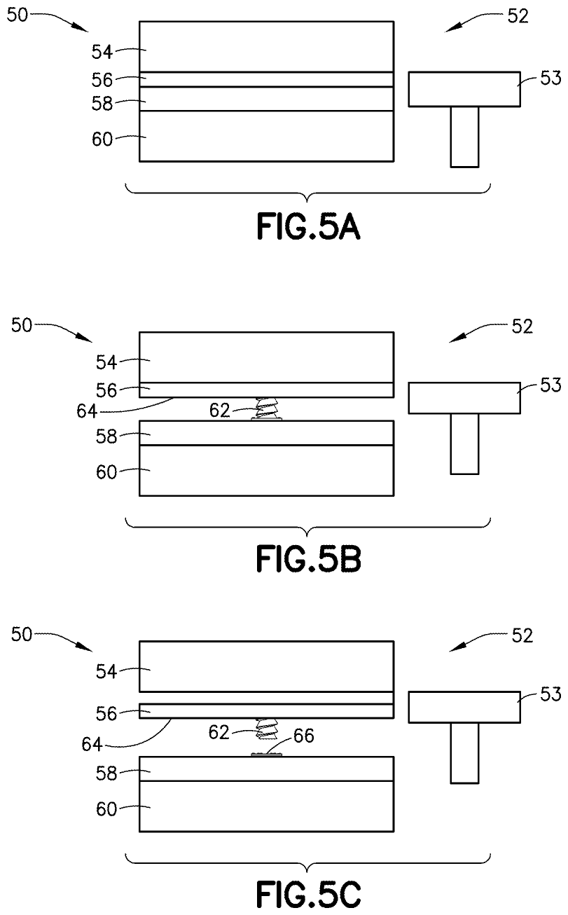

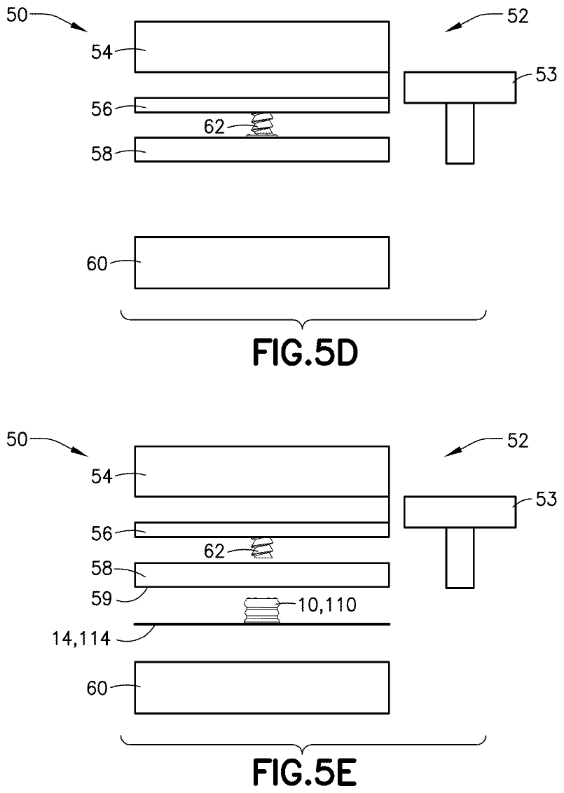

Reference is now made to FIGS. 5A-5E, which illustrate the sequential steps of molding a device 10, 110 using the four-plate injection mold system 50 of the invention. With reference to FIG. 5A, the method includes providing a four-plate mold assembly 52 having top plate 54, a sprue/vent plate 56 including a core portion 62, a main plate 58 including a recess 66 defining a mold cavity 68 having a contoured surface 70, and a bottom plate 60, closing the mold assembly 52 such that the core portion 62 enters into the recess 66 in the main plate 58 to define a space 72, as shown in FIG. 3, corresponding to the shape of the device 10, 110 to be molded, and introducing a polymeric material into the mold cavity 68 to fill the mold cavity 68 and the space to form the molded device 10, 110. The mold assembly 52 can include an optional component 53 that includes a pin shaped member (not shown) that can be held by hand by the operator to the operator to push parts out of the cavity plate 58.

The molded device will have a body 18, 118 and molded outer surface corresponding to the contoured surface 70 of the mold cavity 68. Depending upon the type of polymeric material being used, such as with the use of a thermoplastic elastomer, the method can includes the step of cooling the polymeric material. If the polymeric material comprises an uncured rubber, after the material is introduced into the mold cavity, the mold is heated, such as to approximately 180° C. to cure the rubber. After the polymeric material is cooled or after the rubber is heated to cure, the sprue/vent plate 56 is separated from the main plate 58, as shown in FIG. 5B, to withdraw the core portion 62 from within the molded cavity 68, the recess 66, and the molded device 10, 110. Next, the sprue/vent plate 56 is separated from the top plate 54, as shown in FIG. 5C. Then, as shown in FIG. 5D, the bottom plate 60 is separated from the main plate 58. The molded device or stopper 10, 110 can then be removed, such as from the bottom 59 of the main plate 58, as shown in FIG. 5E.

The step of separating the sprue/vent plate 56 from the main plate 58 causes the vents 57, as shown in FIG. 4, to break and the core portion 62 to be withdrawn from within the molded device 10, 110 and the main plate 58. It can be appreciated that the core portion 62 can be designed to have several shapes depending upon the desired internal shape of the device or stopper 10, 110. For example, the core portion can have a threaded design, a cone-shaped portion with an enlarged portion toward the intersection of the core portion with the sprue/vent plate, a straight sidewall, and the like. Upon opening of the plates of the mold assembly 52, the molded device 10, 110 can be removed from the main plate 58 by pulling the molded device through the bottom 59 of the main plate 58.

Referring back to FIG. 3, the step of introducing the polymeric material into the mold cavity comprises injecting the polymeric material into the mold cavity 68 through an injection nozzle 74 and runner 76 associated with the top plate 54. The polymeric material can be synthetic rubber, however, it can be appreciated that other materials can be used, such as natural rubber, elastomers, and combinations thereof. Examples of elastomers that can be used for the polymeric material include, but are not limited to, silicone rubber, natural rubber, styrene butadiene rubber (SBR), ethylene-propylene diene monomer EPDM), polychloroprene, and the like. If the polymeric material comprises a elastomer, the material is heated, depending on the material but typically it is heated to approximately 225°. Alternatively, if the material comprises, for example, a thermoset rubber, then the material is heated only to where it becomes molten (i.e., around 20-50°) such that it can be injected into the mold cavity. The step of cooling the mold assembly, when necessary, includes supplying a cooling medium, such as via cooling tubes 78, to the main plate 58.

Referring back to FIGS. 1A-1B and 2A-2B, the contoured surface of the main cavity can be in the shape of a stopper 10, 110 having a main body 18, 118 defining an open rearward end 22, 122 a closed front or distal end 20, 120, and a cylindrical sidewall 24, 124 extending between the open rearward end 22, 122 and the closed front or distal end 20, 120. According to one embodiment, the shape of the contoured surface is configured to create a plurality of annular ribs 12, 112 extending radially outward around a perimeter of the main body 18, 118 and axially spaced apart along this main body 18, 118. It can be appreciated that the main cavity can have other contoured surfaces to make other products and/or to create stoppers 10, 110 having different shapes.

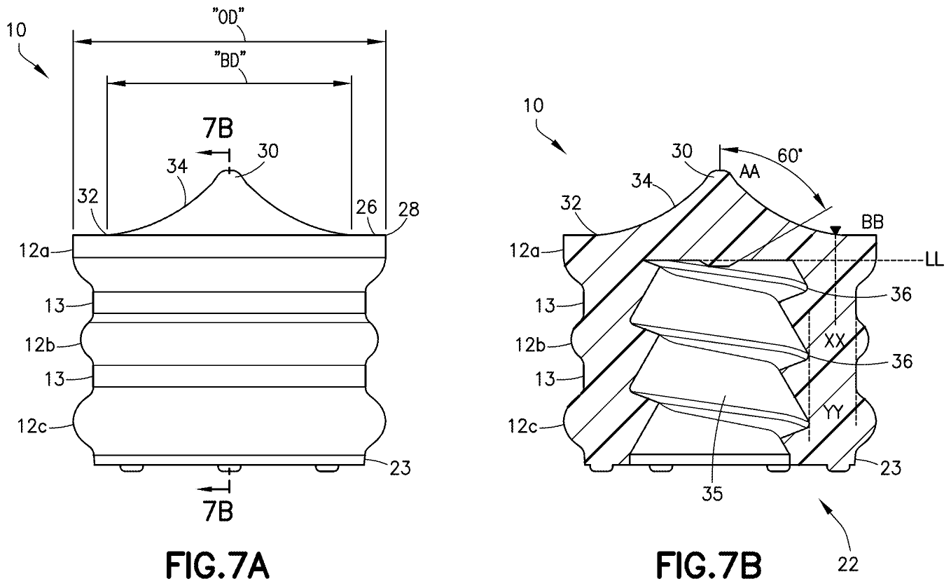

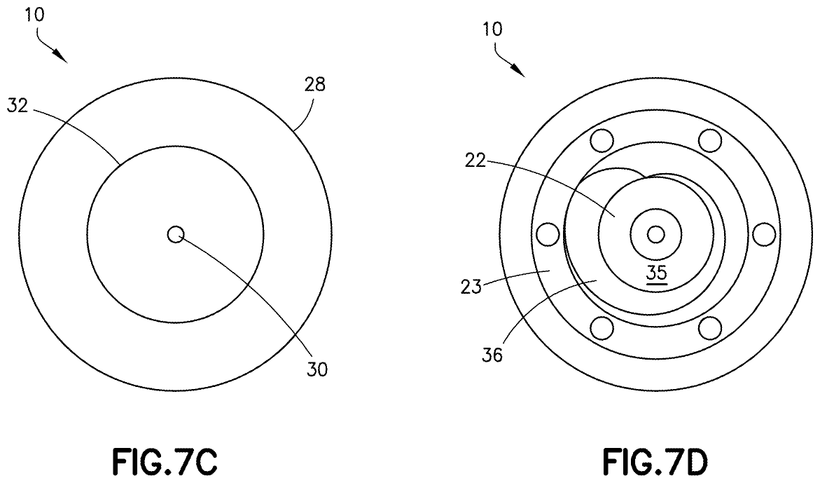

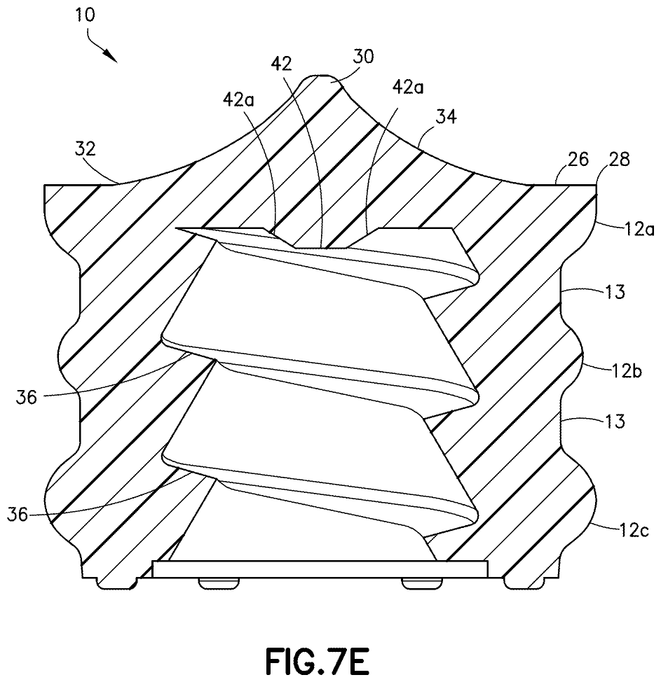

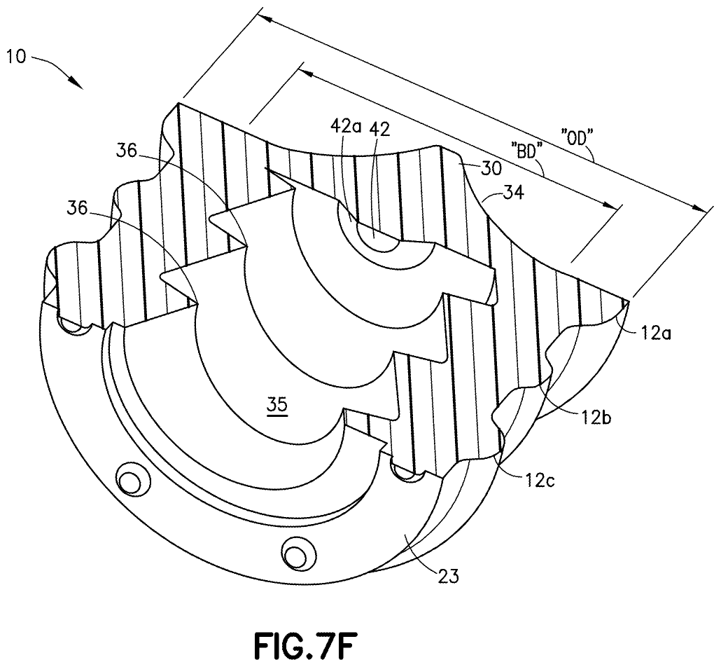

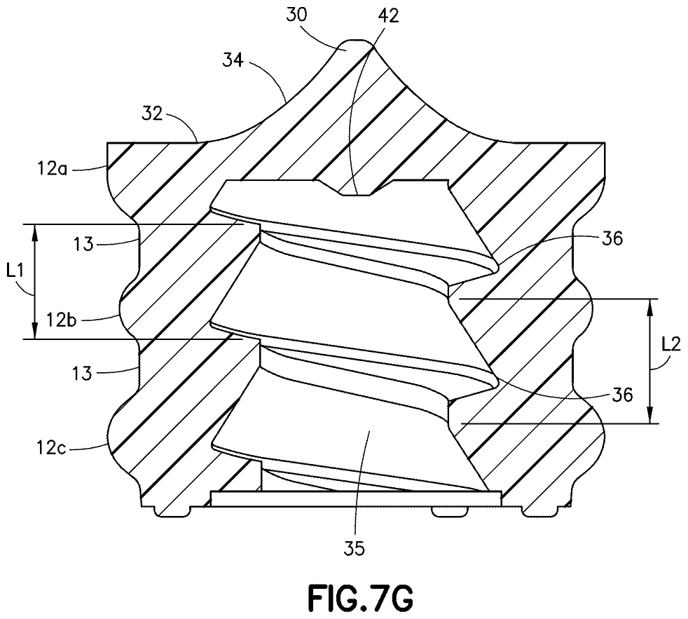

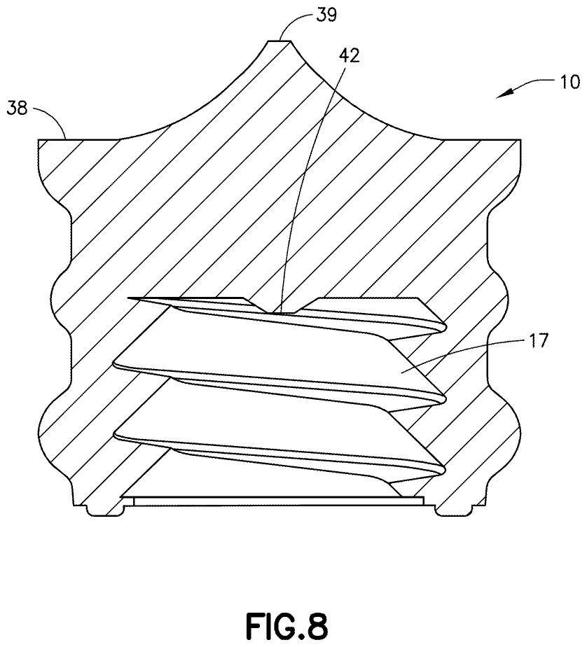

According to one embodiment, the bottom plate 60 can have a flat face configured to create a stopper 110 having a flat face 121 at its distal end 120, as shown in FIGS. 2A and 2B. According to another embodiment, the bottom plate 60 can include a contoured surface configured to create a stopper 10 having distal end 20 as shown in FIGS. 1A and 1B. The overall shape of the stopper of FIGS. 1A and 1B is discussed in more detail below in the description of FIGS. 7A-7G. However, the bottom plate 60 has a contoured surface configured to produce a stopper having a distal end 20 in the shape of a flat portion 26 on an outer edge 28 of the closed front or distal end 20 of the stopper 10 that extends inward from the outer edge 28 of the stopper 10 and a projection 30 extending from the closed front or distal end 20, wherein the projection 30 defines a base 32 as it intersects with the closed front or distal end 20 of the stopper and includes sloped portion 34 that extends in an upward direction from the flat portion 26 of the top of the closed front or distal end 20 to the projection 30. The sloped portion 34 is discussed in more detail below in relation to FIG. 7B. The projection is configured to cooperate within a syringe barrel 16 to reduce dead space during injection. It can be appreciated that the bottom plate 60 can have other designs to create stoppers having a variety of shapes at their distal end.

The method can further include the step of providing a film, such as a barrier film 14, 114, between the bottom plate 60 and the main plate 58 and wherein upon closing of the mold assembly 52, the film 14, 114 is sandwiched between the bottom plate 60 and the main plate 58 upon removal of the device from the mold assembly 52, the film 14, 114 is secured to the molded device. It can be appreciated that the barrier film 14, 114 can be a known film that provides a low friction barrier between, for example, a stopper 10, 110 and a pharmaceutical composition (e.g., a drug, medicine, or other therapeutic material) in the syringe barrel 16 and may inhibit materials from leaching from the stopper 10, 110 or from extraction of compounds from the pharmaceutical composition by the stopper 10, 110.

With reference to FIGS. 6A and 6B, the method further includes the step of trimming and removing excess film 14, 114 from the molded device 10, 110. This trimming step can be accomplished with a trim die 84. According to one design, the trim die can comprise a flat faced punch 86 for removing the excess film 14, 114. The trim die 84 includes a punch center having an opening 88 through which a clean air blast can move therethrough to clear trimmed parts from the die block 85. The flat faced punch 86 has a flat surface 87 for removing the excess film 14, 114. A center portion 90 in the punch 86 is deep enough to allow numerous resharpenings, which is more cost effective than using die punches 86 having a razor edge (which is used when stopper does not have a flat portion, but rather cone slope starts from edge and top rib of the stopper). The razor edge wears out faster, requiring the punches to be resharpened individually, which can be cost prohibitive. However, it can be appreciated that other types of dies having other types of cutting surfaces, such as a razor edge, and/or other types of trimming devices can be used for removing the excess film.

With continuing reference to FIGS. 1A and 1B and with further reference to FIGS. 7A-G, there is shown one type of stopper 10 that can be produced using the injection molding system 50 of the present disclosure. The stopper 10 is adapted for attachment with an attachment end 17 of a plunger rod for use within a syringe barrel 16, as shown in FIGS. 9A-9B.

As discussed above, the stopper 10 comprises a main body 18 defining an open rearward end 22, a closed front or distal end 20, and a cylindrical sidewall 24 extending between the open rearward end and the closed front or distal end 20. A barrier film 14 (not shown in FIGS. 7A-7G) can be provided adjacent at least the closed front or distal end 20. The open rearward end 22 is adapted to receive the front forward end attachment portion 17 of the plunger rod. The stopper 10 further includes at least one rib 12 extending radially outward around a perimeter of the main body. The at least one rib 12 is configured for forming an active seal with the syringe barrel 16. The annular or circumferentially extending ribs 12 are intended to provide a stable fluid-tight seal between the stopper body and the syringe barrel. It can be appreciated that one or more ribs can be provided. In the embodiment disclosed in FIGS. 7A-7G, the main body 18 can include a first, second, and third rib 12a, 12b, and 12c extending radially outward around a perimeter of the main body and axially spaced apart along the main body 18.

The ribs 12 include recesses 13 therebetween wherein the ribs 12 have an outside diameter that is greater than an outside diameter of the recesses 13. The ribs 12 can be curvilinearly shaped when viewed from a distal end of the stopper body 10. It can be appreciated that other embodiments of the stopper can be formed using the molding system 50 of the present disclosure, including those having a smooth cylindrical side surface.

The open rearward end 22 of the stopper 10 comprises a cavity 35 that is configured for receiving the front attachment end 17 of the plunger rod. According to the embodiment shown in FIGS. 7A-7G, this cavity 35 includes threads 36 configured to cooperate with threads 17a on the front attachment end 17, as shown in FIGS. 9A and 9B, of the plunger rod. It can be appreciated that there are many ways to join a plunger rod to a stopper 10, 110 and that the threaded arrangement is exemplary of many possibilities. For example, the cavity 35 in the stopper 10 may have a reduced diameter or neck at the proximal end of the cavity 35 and/or at the open rearward end 22 of the stopper 10. According to one arrangement, this cavity 35 could be shaped to accept a tip or projection 30 on the distal or front attachment end 17 of the plunger rod which has an enlarged distal end so that the parts will come together in a snap-fit arrangement. According to another example, the stopper 10 may be adhesively attached or co-molded to the attachment end 17 of the plunger rod.

With continuing reference to FIGS. 7A-7G, a flat portion 26 is provided on a top outer edge surface of the closed front or distal end 20 of the stopper 10 adjacent the at least one rib 12. The flat portion 26 extends inward from the outer edge 28 of the stopper 10. According to one embodiment, the flat portion 26 can be approximately 0.75-1 mm wide until the start of an upward slope 34 forming the cone-shaped protrusion 30 with an apex at the top of the closed front or distal end 20 of the stopper 10. The projection 30 defines a base 32 as it intersects with the closed front or distal end 20 of the stopper 10 and extends from the flat portion 26 to the top of the closed front or distal end 20. The base 32 has a base diameter “BD” less than an outside diameter “OD” of the closed front or distal end 20 of the stopper 10 and the projection 30 has a profile configured to cooperate with an internal surface 40 of the syringe barrel, as shown in FIGS. 9A and 9B to reduce dead space during injection.

With particular reference to FIGS. 7B, 7E and 7F the stopper 10 of the present disclosure can be designed such that the second rib 12b is slightly smaller in diameter (up to 4-15%) than the other ribs 12a and 12c for the purpose of reducing stopper gliding force while still maintaining a seal. The rib radius of the second rib 12b, at its apex, can be approximately 0.45-0.65 mm, depending on the diameter of the barrel, resulting in lower glide force. A plunger rod cavity protrusion 42 can be provided in bottom of the cavity 35 of the stopper 10. This cavity protrusion 42 is designed to lightly engage the front attachment end 17 of the plunger rod but quickly increase the force transmitted to the front of the stopper (versus transmitting axial load through the threads) in order to pull the front rib 12a first and break the stopper 10 out more easily (reduced initial break loose force). The sidewall 42a of the protrusion 42 is angled steeply, such as at an approximate 60 degree angle with respect to a vertical line extending through the tip of the stopper 10, to make light initial contact with the front attachment end 17 of the plunger rod but increase in force quickly. With continuing reference to FIG. 7B, a thickness between the roof profile spline AA-BB and an internal roof projection LL can be monotonic decreasing and project upward to point XX. Point XX can be located between 0.25-0.75 mm within the thickness of the stopper wall defined by YY. This particular design avoids buckling of the roof in a downward direction at a location of the stopper having a thinner cross section and also prevent possible wrinkling of barrier film 14.

As illustrated in FIG. 7G, the pitch of the threads in the stopper can be variable and non-constant such that it increases toward the rearward end or proximal face 22 of the stopper and/or toward the bottom of the plunger rod cavity 35. This puts additional pressure initially on the front of the stopper 10 to concentrate the applied thumb force through the plunger rod to break loose the first rib 12a of the stopper. This also causes the stopper ribs to break free of the container wall sequentially by pulling it instead of a pusher acting on the rearward end or proximal face 22 of the stopper 10. For example, as illustrated in FIG. 7G, a first pitch L1 can be shorter in length than a second pitch L2. According to one example, the pitch can go from L2 to L1 within the range of 2.1 mm to 1.4 mm. For example, the pitch from L2 to L1 can be reduced in about two turns, wherein L2 is approximately 1.73 mm and L1 is approximately 1.57 mm.

FIG. 8 shows a stopper 10 secured to an attachment end 17 of a plunger rod that can be created using the four-plate injection molding system 50 of the invention. It can be appreciated that this stopper 10 can be formed with or without the barrier film 14, 114. FIGS. 9A and 9B show the interaction of the stopper design of FIG. 8 within a syringe barrel 16 after assembly and after the application of 10 lbf (pound force) or 44.5N to the plunger rod. FIG. 8 is similar in design with the stopper 10 of FIGS. 7A-G. As shown in FIG. 9B, these stoppers result in the least amount of dead space within the syringe barrel 16 after application of 10 lbf (44.5N) to the plunger rod.

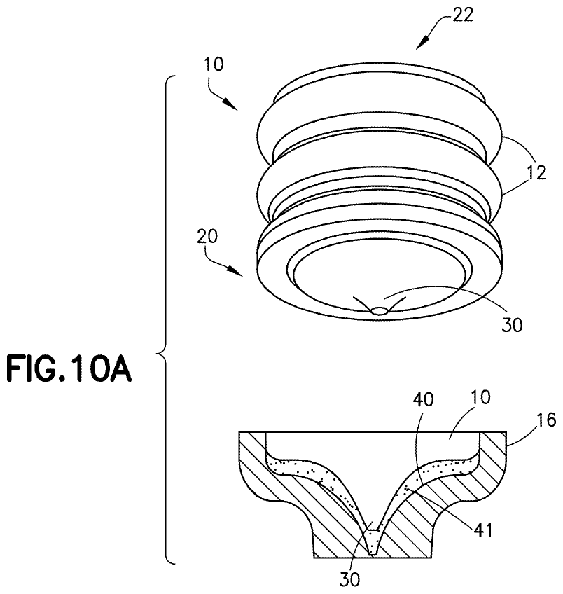

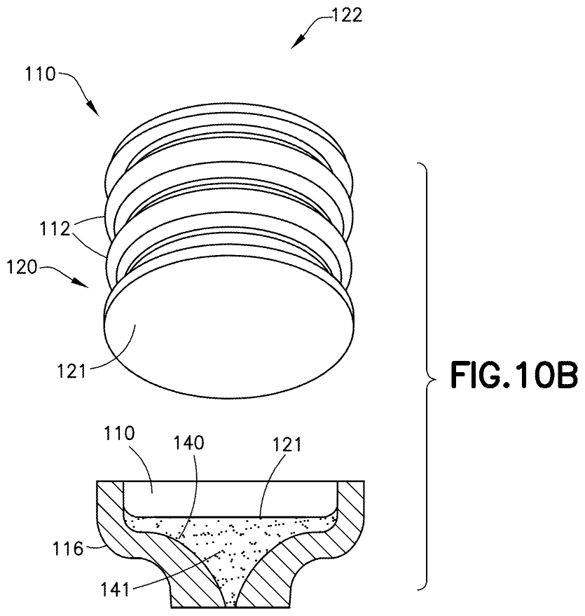

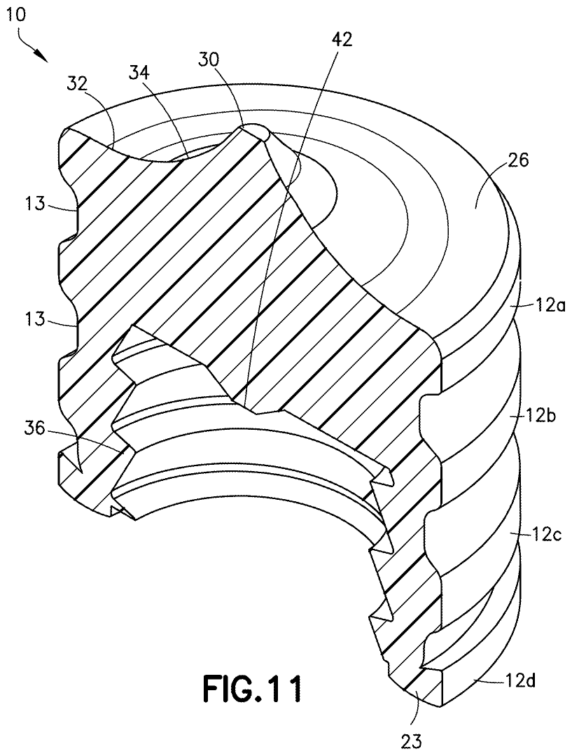

With reference to FIGS. 10A and 10B, it has been found that the stopper 10 of FIG. 10A, having a conical head or cone-shaped protrusion 30 at its distal end 20, results in a significant decrease in dead space at the end of an injection when compared with the stopper 110 of FIG. 10B having a flat face or flat head 121 at its distal end 120. The dead space results in the trapping of medication or drug between the distal end 20, 120 of the stopper 10, 110 and the internal surface 40, 140 at the distal end of the syringe barrel 16, 116. The trapped medication or drug, often referred to as the dead volume, is represented by 41 in FIG. 10A and 141 in FIG. 10B. This dead volume 41, 141 stays in the syringe at the end of the injection and cannot be injected no matter how high of a force is exerted on the plunger rod at the end of the injection. The flat head stopper 110, shown in FIGS. 2A-2B and 10B, is generally used with pre-fillable syringes having a size range of 1-3 ml. However, a finite element analysis (FEA) was conducted to compare the dead volume 41 of the conical head stopper 10 of FIGS. 1A-1B and 10A with the dead volume 141 of the flat head stopper 110 of FIGS. 2A-2B and 10B. The results of this analysis is shown in Table 1 below. A typical force applied to stopper is usually between 10-25N, however, Table 1 also shows values above (44.5N) and below (8.9) this typical range.

TABLE 1

| Estimation of the | Force Applied on the stopper (N) | | | | | | | | | |
|---|---|---|---|---|---|---|---|---|---|---|
| dead volume (mm³) | 8.9N | | 15N | | 22N | | 25N | | 44.5N | |
| 1. Contact Head Stopper | 4.5 | mm³ | 4.5 | mm³ | 4.5 | mm³ | 4.5 | mm³ | 4.5 | mm³ |
| 2. Flat Head Stopper | 18.2 | mm³ | 16.5 | mm³ | 14.9 | mm³ | 14.4 | mm³ | 11.5 | mm³ |

As can be seen in Table 1, the conical head stopper 10 has a dead volume 41 of approximately 4.5 mm³, which is significantly less than the flat head stopper 110, where the dead volume 141 ranges from approximately 11.5 mm³ to 18.2 mm³.

Reference is now made to FIG. 11, which shows a stopper design including a fourth rib 12d provided at the base 23 of the stopper 10. This design includes a thicker front section 46, than the design of FIGS. 7A-7G. This thicker front section 46 helps to reduce buckling of the stopper 10. This particular design, like the design of FIGS. 7A-7G includes a distal end 20 in the shape of a flat portion 26 on an outer edge 28 of the closed front or distal end 20 of the stopper 10 that extends inward from the outer edge 28 of the stopper 10 and a projection 30 extending from the closed front or distal end 20, wherein the projection 30 defines a base 32 as it intersects with the closed front or distal end 20 of the stopper and includes sloped portion 34 that extends in an upward direction from the flat portion 26 of the top of the closed front or distal end 20 to the projection 30. As discussed above in relation to FIG. 10A, the projection 30 is configured to cooperate within a syringe barrel 16 to reduce dead space during injection.

While the disclosure has been described as having exemplary designs, the present disclosure can be further modified within the spirit and scope of this disclosure. This application is, therefore, intended to cover any variations, uses, or adaptations of the disclosure using its general principles. Further, this application is intended to cover such departures from the present disclosure as come within known or customary practice in the art to which this disclosure pertains and which fall within the limits of the appended claims.

The invention claimed is:

1. A method of molding a device comprising the steps of:
providing a mold assembly having top plate, a sprue/vent plate including a core portion, a main plate including a recess defining a mold cavity having a contoured surface, and a bottom plate;
closing the mold assembly such that the core portion enters into the recess in the main plate to define a space corresponding to the shape of the device;
introducing a polymeric material into said mold cavity to fill said mold cavity and said space to form said molded device having a body and molded surface corresponding to said contoured surface;
separating the sprue/vent plate from the main plate to withdraw the core portion from within the molded device;
followed by separating the sprue/vent plate from the top plate;
followed by separating the bottom plate from the main plate; and
removing said molded device from the main plate.

2. The method of claim 1, wherein the step of separating the sprue/vent plate from the main plate causes the vents to break and the core portion to be withdrawn from within the molded device and the main plate.

3. The method of claim 1, wherein the step of removing the molded device from the main plate comprises removing the molded device through the bottom of the main plate.

4. The method of claim 1, wherein the step of introducing the polymeric material into the mold cavity comprises injecting a polymeric material into the mold cavity through an injection nozzle and runner associated with the top plate.

5. The method of claim 1, wherein after the step of introducing polymeric material into the mold cavity, the mold assembly is heated to cure the polymeric material or the mold assembly is cooled by supplying a cooling material to the main plate.

6. The method of claim 1, wherein the contoured surface of the main cavity is in the shape of a stopper having a main body defining an open rearward end, a closed front end, and a cylindrical sidewall extending between the open rearward and the closed front end.

7. The method of claim 6, wherein the shape of the contoured surface is configured to create a plurality of ribs extending radially outward around a perimeter of the main body and axially spaced apart along this main body.

8. The method of claim 6, wherein the bottom plate has a flat face configured to create a stopper having a flat face or the bottom plate includes a contoured surface configured to create a stopper having a flat portion on an outer edge of a closed front end of the stopper and extending inward from the outer edge of the stopper and wherein the contoured surface is shaped to create a projection extending from the closed front end, wherein the projection defines a base as it intersects with the distal end of the stopper and extends from the flat portion of the top of the closed front end, said projection being configured to cooperate within a syringe barrel to reduce dead space during injection.

9. The method of claim 1, including the step of providing a film between the bottom plate and the main plate and wherein upon closing of the mold assembly, the film is sandwiched between the bottom plate and the main plate and upon removal of the device from the mold assembly, the film is secured to the molded device.

10. The method of claim 9, including the step of trimming excess film from the molded device.

11. A system for molding a device comprising:
a mold assembly having top plate, a sprue/vent plate, a main plate, and a bottom plate, the sprue/vent plate including a core portion extending from a surface thereof, the main plate including a recess defining a mold cavity having a contoured surface, said mold cavity configured for receiving the core portion from the sprue/vent plate to define a space corresponding to the shape of the device to be molded;
an injection nozzle and runner mated to the top plate, said injection nozzle and runner configured for supplying a polymeric material into the mold cavity to fill said space to form said molded device having a body and molded surface corresponding to said contoured surface;
at least one cooling member for cooling the polymeric material within the mold cavity;
a first mechanism for separating the sprue/vent plate from the main plate to withdraw the core portion from within the molded device;
a second mechanism for separating the sprue/vent plate from the top plate; and
a third mechanism for separating the bottom plate from the main plate to enable removal of the molded device from the main plate.

12. The system of claim 11, wherein the first, second, and third mechanisms for separating the top plate, sprue/vent plate, main plate, and bottom plate of the mold assembly comprises a series of spring mechanisms of varying force or a series of core lifters to sequentially separate the sprue/vent plate from the main plate: the sprue/vent plate from the top plate; and the bottom plate from the main plate and wherein the spring mechanisms include leader pins to limit the opening of each of the plates.

13. The system of claim 11, wherein a film can be inserted between the bottom plate and the main plate and wherein closing of the mold assembly causes the film to be sandwiched between the bottom plate and the main plate such that the film is secured to the molded device.

14. The system of claim 13, including a trim die with a flat faced punch for removing excess film from the molded device.

15. A stopper adapted for attachment with a plunger rod for use within a syringe barrel, said stopper comprising:
a main body defining an open rearward end, a closed front end, and a cylindrical sidewall extending between the open rearward end and the closed front end, said open rearward end adapted to receive a front forward end attachment portion of the plunger rod;
a barrier film positioned adjacent the closed front end;
at least one rib extending radially outward around a perimeter of said main body, said at least one rib configured for forming an active seal with the syringe barrel;
a flat portion on a top outer edge surface of the closed front end and adjacent the at least one rib, the flat portion extending inward from the outer edge of the stopper; and
a projection extending from the closed front end, wherein the projection defines a base as it intersects with the closed front end of the stopper and extends from the flat portion to the top of the closed front end, said base having a base diameter less than an outside diameter of the distal end of the stopper and said projection having a profile configured to cooperate with an internal surface of the syringe barrel to reduce dead space during injection.

16. The stopper of claim 15, wherein a thickness between a roof profile spline AA-BB and an internal roof projection LL is monotonically decreasing and projects upward to point XX.

17. The stopper of claim 15, wherein point XX is within the range of 0.25-0.75 mm within a thickness of a stopper wall defined by YY.

18. The stopper of claim 15, wherein the open rearward end of the stopper includes a cavity having a series of threads for threaded attachment with an attachment end of the plunger rod and wherein the threads have a pitch that is decreasing toward a bottom of the plunger rod cavity.
